(19) United States
(12) Patent Application Publication
(10) Pub. No.: US 2006/0127351 A1
(43) Pub. Date: Jun. 15, 2006

(54) Process for preparing high molar mass polymers

(75) Inventors: Erin A. S. Doherty, Hockessin, DE (US); Martha G. Hollomon, Newark, DE (US); Robert A. Gelman, Newark, DE (US)

Correspondence Address: HERCULES INCORPORATED;HERCULES PLAZA, 1313 NORTH MARKET STREET, WILMINGTON, DE, 19894-0001, US

(73) Assignee: Hercules Incorporated, Wilmington, DE (US)

(21) Appl. No.: 11/012,010

(22) Filed: Dec. 14, 2004

(51) Int. Cl. A61K 31/74; C08F 132/08; C08F 118/02

(52) U.S. Cl. 424/78.27; 526/281; 526/319

(57) ABSTRACT

The present invention describes processes for preparing water-compatible anionic polymers of high molar mass via water-in-oil polymerization.

### FIELD OF THE INVENTION

The present invention relates generally to processes for preparing water-compatible pH stable anionic polymers of high molar mass via water-in-oil polymerization.

### BACKGROUND

Certain water-soluble anionic polymers of high molar mass are particularly useful in the paper industry. One such polymer is described in copending U.S. Ser. No. ______, filed concurrently, the entire disclosure of which is incorporated herein by reference. Due to their importance, it is desirable to develop effective processes for preparing such polymers.

Previously known anionic polymers were limited to those having relatively low molar mass and/or those that were not selected to maximize retention of charge per unit mass. However, anionic polymers that retain their charge over a wide pH range are particularly useful to accommodate pH variations present in end-use applications. Thus, it is desirable that processes be developed for preparing anionic polymers that retain as much charge per unit mass as possible when the polymer is exposed to lower pH ranges.

The present invention is directed to these, as well as other important ends.

### SUMMARY

In one embodiment, the present invention includes methods for preparing anionic water-compatible (water-soluble or water-dispersible) polymers, the methods comprising preparing an aqueous solution of monomers, wherein at least one of the monomers is A, an ethylenically unsaturated monomer substituted with at least one —S(═O)₂OR₁ or —OS(═O)₂(O)ₚR₁ moiety, wherein R₁ is H, alkyl, aryl, or a cation, and p is 0 or 1; forming an inverse emulsion between the aqueous solution and an organic phase; and polymerizing the monomers to form a pH stable polymer with a weight average molar mass of about 5 million or greater.

### DETAILED DESCRIPTION

In one embodiment, the present invention includes methods for preparing anionic water-compatible polymers (i.e., homopolymers and copolymers). In some embodiments, the present invention includes methods comprising preparing an aqueous solution of monomers, wherein at least one of the monomers is A, an ethylenically unsaturated monomer substituted with at least one —S(═O)₂OR₁ or —OS(═O)₂(O)ₚR₁ moiety, wherein R₁ is H, alkyl, aryl, or a cation, and p is 0 or 1; forming an inverse emulsion between the aqueous solution and an organic phase; and polymerizing the monomers to form a pH stable polymer with a weight average molar mass of about 5 million or greater. Examples of acceptable cations include, but are not limited to, Na⁺, K⁺, Li⁺, NH₄⁺, or alkyl-NH₃⁺, but preferably the cation is sodium or ammonium.

In another embodiment, A is substituted with at least one aryl group in addition to the at least one —S(═O)₂OR₁ or —OS(═O)₂(O)ₚR₁ moiety, but that is not meant to imply that the ethylene moiety must be directly substituted with both the aryl and —S(═O)₂OR₁ or —OS(═O)₂(O)ₚR₁ moiety (see, e.g., Formula IA and IB below). Such an arrangement is a part of the present invention, however, A is also intended to include embodiments where the aryl is attached to the ethylene moiety, and the —S(═O)₂OR₁ or —OS(═O)₂(O)ₚR₁ moiety is attached to the aryl (see, e.g., Formula I below). Likewise, the requirement of the presence of an aryl group can be satisfied by embodiments where R₁ is aryl (see, e.g., Formula IB below).

Examples of monomer A include, but are not limited to, the free acid or salt of: styrenesulfonic acid, vinyltoluenesulfonic acid, α-methyl styrenesulfonic acid, anetholesulfonic acid, vinylphenylsulfonic acid, 4-sulfonate N-benzyl acrylamide, 4-sulfonate N-phenyl acrylamide, vinylpyrenesulfonic acid, vinylanthracenesulfonic acid, vinylpyridiniopropane sulfonate, 2-acrylamido-2-methyl-propanesulfonic acid (AMPS), vinylsulfonic acid, vinylphenylsulfuric acid and mixtures thereof.

In a further embodiment, A can be represented by Formula I: 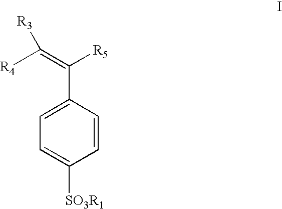
wherein:

R₁ is Na⁺, K⁺, Li⁺, NH₄⁺, or R₅NH₃⁺; and

R₃, R₄, and R₅ are, independently, H or alkyl.

In some embodiments, the monomer A comprises a salt of styrenesulfonic acid. Preferably, the salt is sodium or ammonium salt.

Alternatively, a further embodiment, A can be represented by Formula IA or IB: 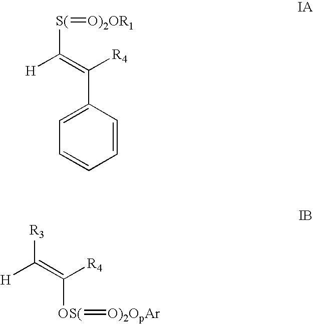
wherein R₃, R₄, and R₅ are, independently, H or alkyl, and Ar is aryl.

It can readily be appreciated that polymers of the present invention can be homopolymers, i.e., entirely comprised of polymer segments formed from ethylenically unsaturated monomer A. A preferred homopolymer includes polymeric segments having Formula II: 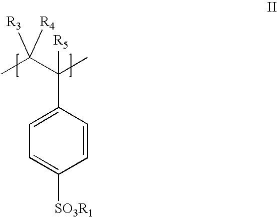
In some preferred embodiments, R₁ is Na⁺.

In another embodiment, the present invention also encompasses copolymers including polymer segments of differing monomers A, as listed above.

In another embodiment, the present invention also encompasses copolymers including polymer segments of monomer A and a polymer segment formed from at least one ethylenically unsaturated anionic, cationic or nonionic monomer (B). It is understood that the term copolymer is not meant to be limiting, and includes all possible monomer sequences involving A and B, or different A monomers and/or B monomers, including random, block, and alternating sequences.

Examples of the monomer B include, but are not limited to, acrylamide, methacrylamide, N-alkylacrylamide, N-methylacrylamide, N,N-dialkyl acrylamide, N,N-dimethylacrylamide, acrylonitrile, N-vinyl methylacetamide, N-vinyl methyl formamide, N-vinylpyrrolidone, alkyl acrylamide, alkyl methacrylamide and mixtures thereof. Additional examples of monomer B include, but are not limited to the free acids or salts of: vinyl acetate, alkyl acrylate, alkyl methacrylate, alkoxylated acrylate, methyl acrylate, methyl methacrylate, methacrylate, alkyl polyethyleneglycol acrylate, alkyl polyethyleneglycol methacrylate, (meth) acrylic acid, maleic acid, fumaric acid, itaconic acid, or acrylamidoglycolic acid, and mixtures thereof. While any anionic, cationic or nonionic monomer that allows the polymer to remain water-compatible is contemplated, preferably, the monomer B is acrylamide or a free acid or salt of acrylic acid. Examples of acceptable salts include those having Na⁺, K⁺, Li⁺, NH₄⁺, or R₅NH₃⁺, but preferably the salt is a sodium or ammonium salt. Monomer B can also optionally be a cationic monomer. Examples of cationic monomers, include, but are not limited to, ethylenically unsaturated quaternary ammonium salts including [2-(acryloyloxy)ethyl]trimethylammonium chloride and the like.

In one embodiment, the molar ratio of A:B is from about 5:95 to about 100:0. In another embodiment, the molar ratio of A:B is from about 5:95 to about 95:5. In another embodiment, the molar ratio of A:B is from about 10:90 to about 90:10. In another embodiment, the molar ratio of A:B is from about 20:70 to about 80:20. It is understood that all combinations and subcombinations of A:B ranges are contemplated.

The total level of cationic monomer is preferably less than 20 mol %, based on the total number of moles of monomer, even more preferably under 15 mol %, and most preferably under 10 mol %.

One preferred embodiment includes copolymers where the monomer A comprises a sodium or ammonium salt of styrenesulfonic acid and the monomer B is acrylamide. A preferred polymer of such embodiments includes polymeric segments having Formula II and Formula III, respectively: 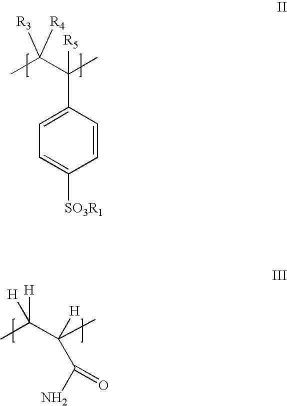
In some preferred embodiments, R₁ is Na⁺.

Another preferred embodiment includes copolymers where the monomer A comprises a sodium or ammonium salt of styrenesulfonic acid and the monomer B comprises a salt of acrylic acid. A preferred polymer of such embodiments includes polymeric segments having Formula II and Formula IV, respectively: 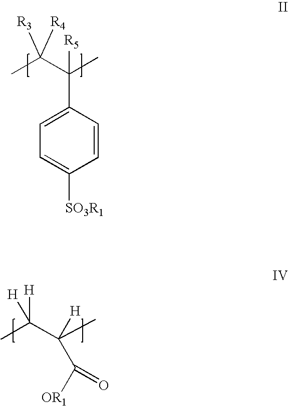
In some preferred embodiments, R₁ is Na⁺ at each occurrence.
Polymerization Process

In one embodiment, an inverse emulsion polymerization process is conducted by preparing an aqueous solution of the selected monomers. The aqueous phase may also contain such conventional additives as are desired. For example, the mixture may contain chelating agents, pH adjusters, initiators, chain transfer agents, and other conventional additives. In some embodiments, chain transfer agents may be used to control the molar mass. Those include, but are not limited to, lower alkyl alcohols such as isopropanol, amines, mercaptans such as mercaptoethanol, phosphites, thioacids, allyl alcohol, and the like. Preferably, the aqueous phase is warmed to dissolve the monomers. In one embodiment, the phase is warmed to about 35-45° C.

Polymerization can occur at any pH basic, neutral or acidic. In one embodiment, the pH of the aqueous phase is equal to or greater than 7. Preferably, the pH of the aqueous phase is below 13, and more preferably about 9 to about 11. However, it is understood that the process would still function with the pH of the aqueous phase as low as about 4 or greater.

The aqueous phase is contacted with an organic phase. Preferably, the aqueous phase is added to the organic phase under an amount of agitation sufficient to disperse sub-micron size aqueous droplets in the organic phase. The organic phase comprises a hydrocarbon liquid. Examples of the hydrocarbon liquid include, but are not limited to, straight-chain hydrocarbons, branched-chain hydrocarbons, saturated cyclic hydrocarbons, aromatic hydrocarbons, and mixtures thereof.

Preferably, the organic phase further comprises surfactant. In preferred embodiments, the surfactants are oil-soluble. Exemplary surfactants include, but are not limited to, sorbitan monooleate, sorbitan sesquioleate, sorbitan trioleate, polyoxyethylene sorbitan monooleate, polyoxyethylene sorbitan hexaoleate and diblock or triblock copolymers based on polyester derivatives of fatty acids and poly(ethylene oxide), diblock and triblock copolymers based on poly(ethylene oxide) and poly(propylene oxide), or mixtures thereof. Preferably, one or more surfactants are selected in order to obtain an overall HLB (Hydrophilic Lipophilic Balance) value ranging from about 2 to 11, preferably about 2 to 8, preferably about 3 to 7, and more preferably about 4 to 6.

The amount (based on weight percent) of total surfactant is dependent on the amount of monomer used. Generally, an amount of surfactant sufficient to provide a stable emulsion for polymerization is used. In one embodiment, the ratio of total surfactant to monomer is at least about 3 to 100. The ratio of total surfactant to monomer can be greater than 3 to 100 and preferably is at least about 4 to 100 and more preferably at least about 5 to 100 and even more preferably at least about 6 to 100.

An inverse monomer emulsion is formed by conventional means, preferably followed by an inverse (water-in-oil) emulsion polymerization technique, such as disclosed in U.S. Pat. No. 3,284,393 and Reissue U.S. Pat. Nos. 28,474 and 28,576, herein incorporated by reference in their entireties. Polymerization of the inverse emulsion may be carried out in any manner known to those skilled in the art. Initiation may be effected with a variety of thermal and redox free-radical initiators or by photochemical irradiation processes, irradiation or by ionizing radiation with a ⁶⁰Co source.

In one embodiment, the monomer emulsion is subjected to free radical polymerization. The free-radical polymerization may be carried out at any pH. Oil-soluble thermal initiators can be used. Typical examples include, but are not limited to, 2,2′-azobis-(2,4-dimethylpentanitrile), 2,2′-azobisisobutyronitrile (AIBN), 2,2′-azobis-(2-methylbutanonitrile), 1,1′-azobis-(cyclohexanecarbonitrile); benzoyl peroxide, and lauroyl peroxide. Alternatively, redox initiators may be used. Examples of redox initiators include, but are not limited to, sulfur dioxide/sodium bromate, t-butyl hydroperoxide/sodium metabisulfite, cumene hydroperoxide/sodium metabisulfite, and the like.

In one embodiment, the method includes optionally adding a breaker surfactant to the polymeric inverse emulsion to enhance the inversion of the emulsion. Breaker surfactants are additional surfactants that are added to an emulsion or the dilution water to promote inversion.

The polymers of the invention may be provided to the end use application in a number of physical forms. The resulting polymers may be isolated by precipitation in an appropriate organic solvent, such as acetone, and dried to a powder form. The powder can then be easily dissolved in an aqueous medium at a desired concentration. In addition to the original emulsion form, the inventive polymer may also be provided as an aqueous solution, dry solid powder, or dispersion form.

Definitions

As used herein, the term “pH stable” refers to those polymers which, at any concentration in deionized water, retain at least about 75% of their charge when the pH of the solution is 4.5 compared to the charge per unit mass on the polymer in a pH 7 solution.

As used herein the term “water compatible” includes both water soluble and water dispersible polymers.

As used herein, the term “alkyl” includes both branched and straight-chain saturated aliphatic hydrocarbon groups having the specified number of carbon atoms, e.g. methyl (Me), ethyl (Et), propyl (Pr), isopropyl (i-Pr), isobutyl (i-Bu), secbutyl (s-Bu), tertbutyl (t-Bu), isopentyl, isohexyl and the like. When any of the foregoing substituents represents or contains an alkyl substituent group, this may be linear or branched and may contain up to 12 carbon atoms, preferably up to 6 carbon atoms, more preferably 1 or 2 carbon atoms.

The term “aryl” means an aromatic carbocyclic moiety of up to 20 carbon atoms, which may be a single ring (monocyclic) or multiple rings (polycyclic) fused together or linked covalently. Any suitable ring position of the aryl moiety may be covalently linked to the defined chemical structure. Examples of aryl moieties include, but are not limited to, chemical groups such as phenyl, 1-naphthyl, 2-naphthyl, dihydronaphthyl, tetrahydronaphthyl, biphenyl, pyrenyl, anthryl, phenanthryl, fluorenyl, indanyl, biphenylenyl, acenaphthenyl, acenaphthylenyl, and the like.

It is understood that the claims encompass all possible stereoisomers, tautomers, salts, and proforms. Moreover, unless stated otherwise, each alkyl and aryl is contemplated as being optionally substituted.

An optionally substituted moiety may be substituted with one or more substituents. The substituent groups which are optionally present may be one or more of those customarily employed. Specific examples of such substituents include halogen, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups. Typically, 0-4 substituents may be present.

The polymers (both homopolymers and copolymers) of the present invention are preferably not branched or cross-linked polymers. For example, preferably no branching or cross-linking agents are utilized in the preparation.

The present compounds are further described in the following examples.

### EXAMPLES

### Example 1

To a suitable reaction flask equipped with an overhead mechanical stirrer, thermometer, nitrogen sparge tube, and condenser was charged an oil phase of paraffin oil (139.0 g, ESCAID 110 oil, ExxonMobil—Houston, Tex.) and surfactants (3.75 g poly(oxyethylene) sorbitan hexaoleate (CIRRASOL G-1086, Uniqema, New Castle, Del.) and 11.25 g sorbitan monooleate).

An aqueous phase was prepared separately which comprised 50 wt % acrylamide (AM) solution in water (37 g, 40 molar % based on total monomer), styrene sulfonic acid, sodium salt powder (NaSS) (81 g, 60 molar % based on total monomer), deionized water (210.0 g), and VERSENEX 80 (Dow Chemical) chelant solution (0.4 g). The aqueous phase was warmed to about 35-45° C. to dissolve the monomers. The pH of the aqueous solution was approximately 9-11.

The aqueous phase was then charged to the oil phase while simultaneously mixing with a homogenizer to obtain a stable water-in-oil emulsion. This emulsion was then mixed with a 4-blade glass stirrer while being sparged with nitrogen for 60 minutes. During the nitrogen sparge the temperature of the emulsion was adjusted to 57±1° C. Afterwards, the sparge was discontinued and a nitrogen blanket implemented.

The polymerization was initiated by feeding a 3 wt % AIBN solution in toluene corresponding to an initial AIBN charge of 75 ppm on a total monomer molar basis. During the course of the reaction, the temperature of the inverse emulsion was maintained at 57±1° C. Four hours after the initial AIBN charge, a 3 wt % AIBN solution in toluene corresponding to a second AIBN charge of 75 ppm on a total molar monomer basis, was charged into the reactor over ˜30 seconds. Then the batch was held at 57±1° C. for 1.5 hours. The final AIBN charge, a 3 wt % AIBN solution in toluene corresponding to a final AIBN charge of 100 ppm on a total molar monomer basis, was charged into the reactor over ˜30 seconds and heated to 65±1° C. and held over about 0.5 hours. The batch was then cooled to room temperature and the product collected.

The preparation of copolymer Examples 2-7 and comparative Example 8 were conducted according to the method of Example 1, except for changes in composition, as described in the following text.

### Examples 2-4

Examples 2-4 were prepared as described in Example 1 except the monomer ratios were Example 2=50 mol % NaSS/50 mol % AM, Example 3=70 mol % NaSS/30 mol % AM, and Example 4=80 mol % NaSS/20 mol % AM.

### Examples 5-6

Examples 5-6 were prepared as described in Example 1 except the monomer ratios were Example 5=70 mol % NaSS/20 mol % AM/10 mol % [2-(acryloyloxy)ethyl]trimethylammonium chloride (AETAC) and Example 6=70 mol % NaSS/15 mol % AM/15 mol % AETAC. The pH of the aqueous phase prepared for Examples 5 and 6 was between 8 and 10. While these particular polymers exhibited weight average molar masses below 5 million (see Table 2), it should be understood that routine experimentation would allow one skilled in the art to optimize conditions to obtain polymers of these compositions having average molar masses of about 5 million or greater, for example by variation of process variables such as initiator concentration.

### Example 7

Example 7 was prepared as described in Example 1, except the monomer was 100 mol % AMPS. The pH of the aqueous phase prepared for Example 7 was between 7 and 9.

### Example 8

Example 8 was prepared as described in Example 3 except the initiator system was varied as indicated in Table 1. In this Example, the initial oxidant (t-butyl hydroperoxide) charge was fed in one portion while the initial reductant (sodium metabisulfite) charge was fed dropwise to the reactor. The time for the reductant addition was 3.5 hours.

Table 1 shows the redox initiation schedule and concentrations.

TABLE 1

| Time (min) | Component | Action |
|---|---|---|
| 0 | 1.0 mL 0.7 wt % tBHP | charge to the reactor |
| | | over ˜30 seconds |
| 10 | 2.0 mL 0.5 wt % SMBS | charge to the reactor |
| | | at 0.013 mL/min |
| 160 | 2.0 mL 5.0 wt % SMBS | charge to the reactor |
| | | at 0.067 mL/min |
| 190 | — | cool to room temperature, |
| | | recover product |

### Example 9 (Comparative)

Comparative Example 9 was prepared as described in Example 1 except the total wt % monomer was increased to 30% while total organic phase mass remained constant, the comonomer mixture was composed of 50 mol % AM and 50 mol % acrylic acid, and the surfactant package was composed of 1 wt % sorbitan monooleate and 2 wt % HYPERMER® B246SF (Uniqema, New Castle, Del.) (both based on the total weight of the emulsion).

The comparative example contains acrylic acid (AA), an anionic monomer, and AM, a nonionic monomer. Although AA is anionic and therefore gives the final polymer a net negative charge, the polymer is not pH stable because acrylic acid is a weak acid that can be easily neutralized by changes in pH within the range of about 4.5 to about 7.

Table 2 lists the physical properties for the compounds described in the Examples.

TABLE 2

| | M_{w} | meq./g | meq./g | % charge |
|---|---|---|---|---|
| Ex. | (106 g/mol)* | pH 7 | pH 4.5 | retained^{†} |
| 1 | 5.8 | 3.88 | 3.43 | 88.4 |
| 2 | 10.4 | 3.67 | 3.22 | 87.6 |
| 3 | 11.2 | 4.20 | 3.67 | 87.4 |
| 4 | 5.2 | 4.60 | 4.31 | 93.6 |
| 5 | 3.1 | 4.28 | 3.93 | 91.8 |
| 6 | 2.2 | 3.20 | 2.82 | 88.1 |
| 7 | 12.7 | 5.10 | 4.79 | 93.9 |
| 8 | 3.5 | 4.80 | 4.30 | 89.6 |
| 9 | 7.2 | 5.05 | 1.64 | 32.6 |

*obtained by multi-angle laser light scattering (MALLS)
${}^{†} \% charge retained = \frac{meq . / g , pH 4.5}{meq . / g , pH 7} \times 100 \%$

Table 2 indicates that the process of this invention is able to produce polymer having a weight average molar mass above about 5 million. The weight average Molar mass M_{W} was determined by batch multi-angle laser light scattering (MALLS) using a Dawn DSP Laser Photometer Optilab DSP Interferometric Refractometer system (Wyatt Technology, Santa Barbara, Calif.). In MALLS batch mode, several concentrations of polymer solution in 1 M NaNo₃ were analyzed in order to extrapolate light scattering and refractive index data to very low scattering angles and concentrations. Zimm plots were then constructed, utilizing the light scattering data from several polymer concentrations and detection angles, to obtain the weight average molar mass M_{W}. MALLS is an absolute molar mass characterization method in that it does not rely on polymer standards. A single precipitated polymer sample was dissolved in an appropriate solvent at a variety of appropriate concentrations and analyzed in batch mode.

Examples 1-4, made with one pH-stable anionic monomer, NaSS, and one nonionic monomer, AM, retained at least about 75% of their charge when the pH of the solution was lowered to pH 4.5 from pH 7. Charge per unit mass measurements were made by measuring streaming potential. The streaming potential determinations were conducted on a Mutek PCD02 at concentrations of 0.003 wt % by titration with 0.001 N hexadimethrine bromide cationic polyelectrolyte to electro-neutral conditions. Adjustments to solution pH were made with dilute hydrochloric acid or dilute sodium hydroxide, as appropriate. The polymers of Examples 1-4 have a high molar mass and exhibit unique physical characteristics regarding their pH-stability. At a concentration of 0.003 wt % in deionized water, the polymers of Examples 1-8 retain at least about 75% of their charge when the pH of the solution is 4.5 compared to the charge per unit mass on the polymer in a pH 7 solution.

Although example 5-6 (NaSS/AM/AETAC) and Example 8 (NaSS/AM initiated with a redox pair) have molar masses below 5 million, it is understood that the molar mass may be increased by changes in process conditions. These changes include, but are not limited to, decreasing the initiator concentration or increasing the monomer concentration.

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

1. A method of preparing a water-compatible anionic polymer, comprising:
preparing an aqueous solution of monomers, wherein at least one of the monomers is A, wherein A is an ethylenically unsaturated monomer substituted with at least one —S(═O)₂OR₁ or —OS(═O)₂(O)ₚR₁ moiety, wherein R₁ is H, alkyl, aryl, or a cation, and p is 0 or 1;
forming an inverse emulsion between the aqueous solution and an organic phase; and
polymerizing the monomers to form a pH stable polymer with a weight average molar mass of about 5 million or greater.

2. The method of claim 1, further comprising heating the aqueous solution to a temperature at least about the polymerization temperature.

3. The method of claim 1, wherein monomer A is selected from the groups consisting of the free acids or salts of: styrenesulfonic acid, vinyltoluenesulfonic acid, α-methyl styrenesulfonic acid, anetholesulfonic acid, vinylphenylsulfonic acid, 4-sulfonate N-benzyl acrylamide, 4-sulfonate N-phenyl acrylamide, vinylpyrenesulfonic acid, vinylanthracenesulfonic acid, vinylpyridiniopropanesulfonate, 2-acrylamido-2-methyl-propanesulfonic acid, vinyl phenyl sulfuric acid, vinylsulfonic acid, and mixtures thereof.

4. The method of claim 1, wherein A comprises the free acid or salt of styrenesulfonic acid.

5. The method of claim 1, wherein the monomers further comprise an ethylenically unsaturated monomer, B, wherein B contains at least one of acrylamide, methacrylamide, N-alkylacrylamide, N-methylacrylamide, N,N-dialkyl acrylamide, N,N-dimethylacrylamide, acrylonitrile, N-vinyl methylacetamide, alkyl acrylamide, alkyl methacrylamide, N-vinyl methyl formamide, vinyl acetate, N-vinylpyrrolidone, acrylonitrile, a free acid or salt of: methyl acrylate, methyl methacrylate, alkyl acrylate, alkyl methacrylate, alkoxylated acrylate, methacrylate, alkyl polyethyleneglycol acrylate, alkyl polyethyleneglycol methacrylate, (meth) acrylic acid, maleic acid, fumaric acid, itaconic acid, acrylamidoglycolic acid; and [2-(acryloyloxy)ethyl]trimethylammonium chloride.

6. The method of claim 5, wherein B comprises acrylamide and wherein A comprises a free acid or salt of styrenesulfonic acid.

7. The method of claim 5, wherein the molar ratio of A:B is from about 5:95 to about 100:0.

8. The method of claim 5, wherein the molar ratio of A:B is from about 5:95 to about 95:5.

9. The method of claim 1, wherein the pH of the aqueous phase is about 4 or greater.

10. The method of claim 1, wherein the pH of the aqueous phase is equal to or greater than 7.

11. The method of claim 1, wherein the organic phase is selected from the group consisting of straight-chain hydrocarbons, branched-chain hydrocarbons, saturated cyclic hydrocarbons, aromatic hydrocarbons, and mixtures thereof.

12. The method of claim 1, wherein the organic phase includes a surfactant selected from the group consisting of sorbitan monooleate, sorbitan sesquioleate, sorbitan trioleate, polyoxyethylene sorbitan monooleate, diblock or triblock copolymers based on polyester derivatives of fatty acids and poly(ethylene oxide), diblock and triblock copolymers based on poly(ethylene oxide) or poly(propylene oxide), and mixtures thereof.

13. The method of claim 1, wherein the step of polymerizing further comprises contacting the mixture a compound selected from the group consisting of with 2,2′-azobis-(2,4-dimethylpentanitrile), 2,2′-azobisisobutyronitrile, 2,2′-azobis-(2-methylbutanonitrile), 1,1′-azobis-(cyclohexanecarbonitrile), benzoyl peroxide, lauroyl peroxide and mixtures thereof.

14. The method of claim 1, wherein the step of polymerizing further comprises contacting the mixture with sulfur dioxide/sodium bromate, t-butyl hydroperoxide/sodium metabisulfite, or cumene hydroperoxide/sodium metabisulfite.

15. A method of improving retention of charge for water-compatible anionic polymers, comprising:
preparing an aqueous solution of monomers, wherein at least one of the monomers is A, wherein A is an ethylenically unsaturated monomer substituted at least one —S(═O)₂OR₁ or —OS(═O)₂(O)ₚR₁ moiety, wherein R₁ is H, alkyl, aryl, or a cation, and p is 0 or 1;
forming an inverse emulsion between the aqueous solution and an organic phase; and
polymerizing the monomers to form a polymer with a weight average molar mass of about 5 million or greater.

16. The method of claim 15, wherein monomer A is selected from the group consisting of the free acid or salt of: styrenesulfonic acid, vinyltoluenesulfonic acid, α-methyl styrenesulfonic acid, anetholesulfonic acid, vinylphenylsulfonic acid, 4-sulfonate N-benzyl acrylamide, 4-sulfonate N-phenyl acrylamide, vinypyrenesulfonic acid, vinylanthracenesulfonic acid, vinylpyridiniopropanesulfonate, 2-acrylamido-2-methyl-propanesulfonic acid, vinylsulfonic acid; and mixtures thereof.

17. The method of claim 15, wherein the monomers further comprise an ethylenically unsaturated monomer, B, wherein B contains at least one of acrylamide, methacrylamide, N-alkylacrylamide, N-methylacrylamide, N,N-dialkyl acrylamide, N,N-dimethylacrylamide, acrylonitrile, N-vinyl methylacetamide, alkyl acrylamide, alkyl methacrylamide, N-vinyl methyl formamide, vinyl acetate, N-vinylpyrrolidone, acrylonitrile, a free acid or salt of: methyl acrylate, methyl methacrylate, alkyl acrylate, alkyl methacrylate, alkoxylated acrylate, methacrylate, alkyl polyethyleneglycol acrylate, alkyl polyethyleneglycol methacrylate, (meth) acrylic acid, maleic acid, fumaric acid, itaconic acid, acrylamidoglycolic acid; and [2-(acryloyloxy)ethyl]trimethylammonium chloride.

18. The method of claim 15 wherein monomer A comprises a free acid or salt of styrenesulfonic acid.

19. The method of claim 18 wherein monomer B comprises acrylamide.

20. The method of claim 17, wherein the molar ratio of A:B is from about 5:95 to about 95:5.
